(19) United States
(12) Patent Application Publication
(10) Pub. No.: US 2006/0239945 A1
(43) Pub. Date: Oct. 26, 2006

(54) Cleansing composition

(75) Inventors: Mohini Anand Bapat, Mumbai (IN); Prem Chandar, Trumbull, CT (US); Ritu Verma, Singapore (SG); Martin Swanson Vethamuthu, Trumbull, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/315,932

(22) Filed: Dec. 21, 2005

(30) Foreign Application Priority Data
Dec. 24, 2004 (IN) 1403/MUM/2004

(51) Int. Cl. A61K 8/34

(52) U.S. Cl. 424/62

(57) ABSTRACT

A skin-lightening cleansing composition with a pH below 7.5 comprising 3% to 50% by weight of detergent active wherein nonionic detergent active when present, has a HLB in the range 5-18; and 0.01% to 20% by weight alkyl or cyclo alkyl resorcinol.

The present invention relates to a novel system for enhanced delivery of functional ingredients through cleansing compositions. In particular, the invention is directed to enhancing the deposition of benefit agents such as skin-lightening agents onto skin, hair or other substrates from detergent based cleansing compositions.

Resorcinol and its derivatives have a wide variety of applications. The largest consumption of resorcinol is in the tyre industry, where the preferred hardening resins are based on resorcinol. Another value-added application of resorcinol and its derivatives is in cosmetic products. Some compounds like 2-4,dihydroxyacetophenone have been used in sun-protective applications.

Alkyl resorcinols and aromatic resorcinols are reported to possess valuable therapeutic and antiseptic properties. In particular, 4-alkyl resorcinol is reported to have skin-beautifying effect, and low toxicity and irritation when applied on to human skin. 4-Alkyl resorcinol has also been reported to be used to inhibit browning of foods and beverages. Alkyl resorcinols like 4-n-butyl resorcinol have been used in skin creams and lotions which are claimed to have good bleaching and anti-microbial effect. 2-alkyl resorcinol (where the alkyl group is linear) has been reported to have skin depigmentation properties.

Melanin is a polymer synthesised by melanocytes, a cell type present at the dermis-epidermis junction, from the amino acid tyrosine. Tyrosine is acted upon by the enzyme tyrosinase which is the key enzyme in melanogenesis.

In the melanosomes, contained within melanocytes, melanin is synthesised from the individual monomers in the melanocyte. The melanised melanosome gets transferred to the neighbouring cells called keratinocytes. The keratinocytes divide and differentiate, and thus transport the melanosome to the surface of the skin. The intensity of the skin colour is directly related to the number, size, melanin content, and the rate of formation and migration/transfer of melanosomes to the keratinocytes.

WO2004069221 (Warner-Lambert Company LLC, 2004) discloses topical formulation for skin-lightening agents, such as 4-cycloalkyl resorcinols. The formulation disclosed contains a skin-lightening agent in combination with a carrier, where the carrier contains at least one hydroxyl solvent and at least one co-solvent.

US20040120907 and US20040115145 (Unilever, 2004), discloses the skin-lightening benefits of 1,3-dithiane resorcinol compounds. US20040109832 (Unilever, 2004) discloses the skin-lightening benefits of 4,6-di-substituted resorcinol derivatives. However these patents refer to use of the resorcinol derivatives in a cosmetic base, and not in a cleansing composition.

EP0904774 (Pfizer, 2003), discloses 4-cycloalkyl resorcinols for cosmetic purposes, to lighten or reduce the pigmentation of the skin affected by the condition. These resorcinol derivatives are also useful for the treatment of inflammatory disorders such as psoriasis and acne.

Cosmetic compositions to deliver different benefit agents are prepared using different emulsifying systems and vehicles. These compositions are generally formulated as creams, lotions and other forms that are leave on products.

Skin-lightening agents such as resorcinol derivatives and others are generally unstable in alkaline pH. Often personal wash formulations are soap based, and have an alkaline pH, thus providing conditions that prove detrimental to the formulations containing skin-lightening agents. Hence it is a problem to formulate cleansing compositions comprising many skin-lightening agents, and also skin-lightening agents such as resorcinol derivatives in cleansing compositions.

It has now been found that it is possible to formulate a stable cleansing composition comprising alkyl resorcinol where the pH of the formulation is essentially maintained below 7.5 using suitable detergent actives.

It is thus an object of the present invention to be able to formulate a stable skin-lightening cleansing composition where the pH of the formulation is essentially maintained below 7.5 comprising alkyl resorcinol as the active skin-lightening agent.

It is another object of the present invention to be able to formulate stable skin-lightening cleansing compositions comprising alkyl resorcinol as the active skin-lightening agent that is capable of depositing the active on to the skin to give superior efficacy.

Thus according to a first aspect of the present invention, there is provided a skin-lightening cleansing composition with a pH below 7.5 comprising:
i. 3% to 50% by weight of detergent active wherein nonionic detergent active, when present, has a HLB in the range 5-18; and
ii. 0.01% to 20% by weight alkyl or cyclo alkyl resorcinol.

It is also preferred that at least 25% by weight of the detergent active is selected from anionic, cationic, amphoteric or zwitterionic surfactants. It is particularly preferred that there is no free soap in the formulation.

According to the essential part of the invention, it is necessary to maintain the formulation at pH below 7.5 to ensure stability and efficacy of the product, and when nonionic detergent actives are present in the formulation they are selected to have a HLB in the range 5-18. Apart from the essential ingredients defined, other conventional ingredients may be incorporated into the formulation.

Resorcinol derivatives of the general formula (I) are useful as skin-lightening agents:

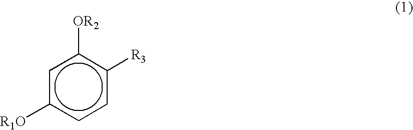

Each R₁ and R₂, independently, represents a hydrogen atom, —COR (acyl group), —COOR, —CONHR; the latter three represented by the following formula A, respectively: 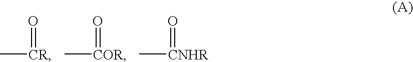
where R represents saturated or unsaturated, linear, branched or cyclic C₁-C₁₈ hydrocarbon groups. In a preferred embodiment, each or both R₁ and/or R₂ represents hydrogen. In a more preferred embodiment, both R₁ and R₂ represent hydrogen.

R₃ represents an alkyl group, having from 1 to 18 carbon atoms, preferably having from 2 to 12 carbon atoms, with or without substitution of one or more hydrogen atoms of a linear alkyl group with a methyl or ethyl group; e.g., R₃ constitutes linear or branched chain alkyls, or a group of the general formula (II): 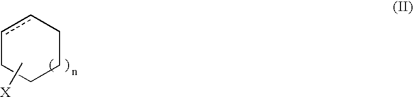
wherein X is hydrogen; OR¹, wherein R¹ represents hydrogen, (C₁-C₆)alkyl or aryl-(C₁-C₆)alkyl; OCOR² wherein R² represents (C₁-C₆)alkyl, aryl-(C₁-C₆)alkyl or phenyl; halogen; (C₁-C₆)alkyl; aryl-(C₁-C₆)alkyl, or aryl-(C₁-C₆) alkyl; or NHR¹ wherein R¹ is defined as above; wherein n is 0 to 3; and wherein the dashed line indicates an optional double bond. For example, where n is 0, the group of general formula (II) is a 5 member ring; where n is 1, the group is a 6 member ring; where n is 2, a 7 member ring; and where n is 3, an 8 member ring.

When R³ represents an alkyl group, the class of molecules are referred to as 4-alkyl substituted resorcinols. In the above formula (1), the unsubstituted linear alkyl group represented by R³ and preferably having from 2 to 12 carbon atoms may include an ethyl group, a propyl group, a butyl group, a pentyl group, a hexyl group, a heptyl group, an octyl group, a nonyl group, a decyl group, an undecyl group and a dodecyl group. These linear alkyl groups may be substituted with a methyl or ethyl group at one or more hydrogen atoms thereof. Specific examples of the substituted alkyl group include an isopropyl group, an isobutyl group, an isoamyl group, or a 2-methylhexyl group. Preferred alkyl groups are those where R³ is an ethyl, propyl, butyl, pentyl, hexyl, heptyl or octyl group. The most preferable alkyl resorcinols are those where R is an ethyl, a butyl or a hexyl group.

The resorcinol derivatives of general formula (I) where R₃ is represented by above formula (II) are referred to herein as 4-cycloalkyl resorcinols and are represented by the general formula (III) as shown below: 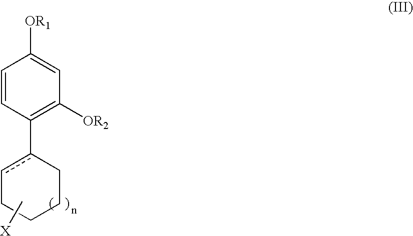
X is hydrogen; OR¹, wherein R¹ represents hydrogen, (C₁-C₆) alkyl or aryl-(C₁-C₆)alkyl; OCOR² wherein R represents (C₁-C₆) alkyl, aryl-(C₁-C₆)alkyl or phenyl; halogen; (C₁-C₆)alkyl; aryl-(C₁-C₆)alkyl, or aryl-(C₁-C₆) alkyl; or NHR¹ wherein R is defined as above;

n is 0 to 3; and the dashed line indicates an optional double bond at that position.

Examples of more specific embodiments of the 4-cyclo-substituted resorcinols include:
(a) compounds of the formula (III) wherein a single bond connects the two carbon atoms at the dashed line;
(b) compounds of the formula (III) wherein n is one;
(c) compounds of the formula (III) wherein X is hydrogen;
(d) compounds of the formula (III) wherein X is hydrogen, methyl or ethyl;
(e) compounds of the formula (III) wherein n is zero;
(f) compounds of the formula (III) wherein n is two; and
(g) compounds of the formula (III) wherein X is benzyloxy.

Preferred compounds of formula (III) are 4-cyclopentylresorcinol, 4-cyclohexyl resorcinol, 4-cycloheptyl resorcinol, and 4-cyclooctyl resorcinol. Most preferred compounds of formula (III) are 4-cyclohexylresorcinol and 4-cyclopentylresorcinol.

The amount of the resorcinol derivative is preferably in the range of about 0.01% to about 20%, more preferably about 0.1% to 10%, most preferably about 0.1% to about 5%, of the total amount of a cosmetic composition.

The detergent active used in the composition is devoid of free soap and comprises non-soap detergents. The detergent active is preferably selected from anionic, nonionic, cationic, amphoteric or zwitterionic surfactans. The total concentration of the detergent active will range from 3% to 50%, preferably from 5% to 25%.

It is particularly preferred that at least 25% by weight of the detergent active is selected from anionic, cationic, amphoteric or zwitterionic surfactants. It is preferred to formulate the detergent actives by selecting a combination of anionic, nonionic and amphoteric surfactants.

Anionic surfactants may be selected from an aliphatic sulfonate, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS), or an aromatic sulfonate such as alkyl benzene sulfonate.

The anionic may also be an alkyl sulfate (e.g C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of greater than 1.0, preferably between 2 and 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, and acyl isethionates.

Sulfosuccinates may be monoalkyl sulfosuccinates having the formula:
R₄O₂CCH₂CH(SO₃M)CO₂M;
amido-MEA sulfosuccinates of the formula:
R⁴CONHCH₂CH₂O₂CCH₂CH(SO₃M)CO₂M
wherein R⁴ ranges from C₈-C₂₂ alkyl and M is a solubilizing cation;
amido-MIPA sulfosuccinates of formula:
RCONH(CH₂)CH(CH₃)(SO₃M)CO₂M
where M is as defined above.

Also included are the alkoxylated citrate sulfosuccinates; and alkoxylated sulfosuccinates such as the following: 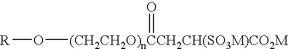
wherein n=1 to 20; and M is as defined above.

Sarcosinates are generally indicated by the formula:
RCON(CH₃)CH₂CO₂M,
wherein R ranges from C₈ to C₂₀ alkyl and M is a solubilizing cation.

Taurates are generally identified by formula:
R²CONR³CH₂CH₂SO₃M
wherein R² ranges from C₈-C₂₀ alkyl, R³ ranges from C₁-C₄ alkyl and M is a solubilizing cation.

Another class of anionics are carboxylates such as follows:
R(CH₂CH₂O)ₙCO₂M
wherein R is C₈ to C₂₀ alkyl; n is 0 to 20; and M is as defined above. Another carboxylate which can be used is amido alkyl polypeptide carboxylates such as, for example, Monteine LCQ (R) by Seppic.

Another surfactant which may be used are the C₈-C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

Acyl isethionates, when present, will generally range from about 0.5% to 15% by weight of the total composition. Preferably, this component is present from about 1% to about 10%.

The nonionic detergent active when present has a HLB in the range 5-18, and can be selected from the reaction products of compounds having a hydrophobic group and a reactive hydrogen atom, for example aliphatic alcohols, acids, amides or alkyl phenols with alkylene oxides, especially ethylene oxide either alone or with propylene oxide. Specific nonionic detergent compounds are alkyl (C₆-C₂₂) phenols-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides. The nonionic may also be a sugar amide, such as a polysaccharide amide.

Alkyl polysaccharide nonionic surfactants may be used. Preferred alkyl polysaccharides are alkylpolyglycosides of the formula:
R²O(CₙH₂ₙO)ₜ (glycosyl)ₓ
wherein R² is selected from the group consisting of alkyl, alkylphenyl, hydroxyalkyl, hydroxyalkylphenyl, and mixtures thereof in which alkyl groups contain from about 10 to about 18, preferably from about 12 to about 14, carbon atoms; n is 0 to 3, preferably 2; t is from 0 to about 10, preferably 0; and x is from 1.3 to about 10, preferably from 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position.

Amphoteric detergents which may be used in this invention include at least one acid group. This may be a carboxylic or a sulphonic acid group. They include quaternary nitrogen, and therefore are quaternary amido acids. They should generally include an alkyl or alkenyl group of 7 to 18 carbon atoms. They will usually comply with an overall structural formula: 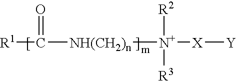
where R¹ is alkyl or alkenyl of 7 to 18 carbon atoms; R² and R³ are each independently alkyl, hydroxyalkyl or carboxyalkyl of 1 to 3 carbon atoms; n is 2 to 4; m is 0 to 1; X is alkylene of 1 to 3 carbon atoms optionally substituted with hydroxyl; and Y is —CO₂ or —SO₃.

Suitable amphoteric detergents within the above general formula include simple betaines of formula: 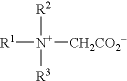
and amido betaines of formula: 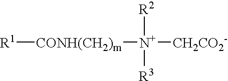
where m is 2 or 3.

In both formulae R , R and R are as defined previously. R¹ may in particular be a mixture of C₁₂ and C₁₄ alkyl groups derived from coconut so that at least half, preferably at least three quarters of the groups R¹ have 10 to 14 carbon atoms. R² and R³ are preferably methyl.

A further possibility is that the amphoteric detergent is a sulphobetaine of formula: 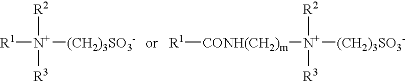
where m is 2 or 3, or variants of these in which —(CH₂)₃SO⁻₃ is replaced by:

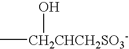

In these formulae R¹, R² and R³ are as discussed previously. Amphoacetates and diamphoacetates are also intended to be covered in possible zwitterionic and/or amphoteric compounds which may be used.

When zwitterionic detergents are present, they may be chosen from derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is: 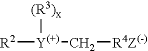
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Examples of such surfactants include:
4-[N,N-di(2-hydroxyethyl)-N-octadecylammonio]-butane-1-carboxylate;
5-[S-3-hydroxypropyl-S-hexadecylsulfonio]-3-hydroxypentane-1-sulfate;
3-[P,P-diethyl-P-3,6,9-trioxatetradexocylphosphonio]-2-hydroxypropane-1-phosphate;
3-[N,N-dipropyl-N-3-dodecoxy-2-hydroxypropylammonio]-propane-1-phosphonate;
3-(N,N-dimethyl-N-hexadecylammonio)propane-1-sulfonate;
3-(N,N-dimethyl-N-hexadecylammonio)-2-hydroxypropane-1-sulfonate;
4-[N,N-di(2-hydroxyethyl)-N-(2-hydroxydodecyl) ammonio]-butane-1-carboxylate;
3-[S-ethyl-S-(3-dodecoxy-2-hydroxypropyl)sulfonio]-propane-1-phosphate;
3-[P,P-dimethyl-P-dodecylphosphonio]-propane-1-phosphonate; and
5-[N,N-di(3-hydroxypropyl)-N-hexadecylammonio]-2-hydroxy-pentane-1-sulfate.

Thickeners may also be utilized as part of the compositions according to the present invention. Typical suitable thickeners include cross-linked acrylates (e.g. Carbopol 982), hydrophobically-modified acrylates (e.g. Carbopol 13 82), cellulosic derivatives and natural gums.

Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. When present amounts of the thickener may range from 0.01% to 20%

Inorganic thickeners may also be employed. These include thickening silicas, chalk, talc, kaolin, starch, smectite clays, chemically modified magnesium aluminum silicate, organically modified montmorillonite clay, laponite, hydrated aluminum silicate, fumed silica, aluminum starch octenyl succinate and mixtures thereof. Amounts of the thickener may range from 0.0001% to 80%, usually from 0.1% to 35%, optimally from 0.5% to 10% by weight.

Humectants of the polyhydric alcohol-type may also be employed as cosmetically acceptable carriers in compositions of this invention. The humectant aids in increasing the effectiveness of the emollient, reduces scaling, stimulates removal of built-up scale and improves skin feel. Typical suitable polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, 10 hydroxypropyl sorbitol, hexylene glycol, 1,3-butylene glycol, 1,2,6-hexanetriol, ethoxylated glycerol, propoxylated glycerol and mixtures thereof.

For best results the humectant is preferably glycerol or isopropyl glycerol. The amount of humectant may range anywhere from 0.01% to 50%, preferably between 1% and 15% by weight of the composition. The particularly preferred humectants are glycerol, isopropyl glycol, polyethylene glycol of different molecular weights.

Suitable additional optional skin benefit agents including anti-aging, wrinkle-reducing, skin-whitening, anti-acne, and sebum reduction agents may be included in the formulation.

The following examples are by way of example only, not by way of limitation, of the principles of the present invention, to illustrate the best mode of carrying out the invention.

### EXAMPLES

### 1. Procedure for Making a Liquid Cleanser

The ingredients as defined in Table 1 and Table 2 were added to water at high speeds in a vortex to completely disperse the same. Preservatives and stabilizers were then added to the main batch. At this point the pH is adjusted to get a final pH as indicated in the Tables 1 and 2. Following this, 4 ethyl resorcinol was added to the batch, and vacuum is used for de-aeration if required. At this point perfume and other optional additives were added.

The formulations were tested for determining various physical and efficacy parameters by the following procedures.

### 2. Procedure for Measuring Product Stability

Stability of product is evaluated after a storage period of 3 months at ˜25° C. by visual measurement to assess color and homogeneity of product.

TABLE 1

| | | |
|---|---|---|
| Composition (% wt.) | | |
| Sodium lauryl sulphate | 7 | — |
| Neutralised fatty Acid | — | 7 |
| (C18/C14/C12 mix) | | |
| 4 ethyl resorcinol (ER) | 2.5 | 2.5 |
| Carbopol ETD 2020 | 0.5 | 0.5 |
| Glycerin | 4 | 4 |
| Sodium Bisulfite | 0.1 | 0.1 |
| Butylated hydroxy toluene | 0.05 | 0.05 |
| Water | to 100 | to 100 |
| pH | 6.5 | 9.2 |
| Product Properties | | |
| Color Stability | Good | Dark brown |
| Homogeneity | Good | Small precipitate |
| | | observed |

The data show that the stability of 4 ethyl resorcinol in the formulation is good when the pH of the formulation is below 7.5.

### 3. Procedure for Measuring Skin Delivery

Delivery of resorcinol on human skin was assessed through direct application of product to human volunteer forearms. A known dose of the product was applied to the area, and the product was rubbed into the area. The area on the arm was rinsed with a fixed amount of water. The collected rinse liquor was analyzed for resorcinol, and the amount delivered to skin was calculated by subtracting from the dosage applied. This is expressed as a percentage of the initial dose.

### 4. Procedure for Measuring Efficacy on Skin-Lightening

Thirty three human panelists are recruited who are required to use a known amount of the cleansing product on one half of their face. At the same time, the other half was rinsed with a placebo cleanser. Panelists were required to do this for eight weeks. The color of the skin is monitored at regular intervals by trained visual graders and instruments. A final assessment is made at eight weeks. The value for skin-lightening indicated is in comparison to the placebo effect.

The data is presented in Table 2.

TABLE 2

| Composition (% wt.) | | | |
|---|---|---|---|
| SLES (3EO) | 6.8 | 6.8 | 4.5 |
| Coca amido propyl betaine | 2.2 | 2.2 | 1.47 |
| Decyl Polyglucoside (HLB 13-15) | — | — | 1.5 |
| 4, ethyl resorcinol (ER) | 2.5 | — | 2.5 |
| Carbopol ETD 2020 | 1.25 | 1.25 | 1.25 |
| Glycerin | 9 | 9 | 9 |
| Sodium Bisulfite | 0.1 | 0.1 | 0.1 |
| Butylated hydroxy toluene | 0.05 | 0.05 | 0.05 |
| Water | to 100 | to 100 | to 100 |
| pH | 6.5 | 6.5 | 6.5 |
| Product Properties | | | |
| Color Stability of ER | Good | Good | Good |
| Skin Delivery of ER | 7.7% | nil | 12.4% |
| Efficacy on skin-lightening | −0.7 | 0 | — |

The data presented in Table 2 show that combination of anionic and amphoteric or in combination with a nonionic surfactant gives significant delivery of 4 ethyl resorcinol from the formulation on the skin, and significantly improves skin colour by lightening it even through a cleansing system.

1. A skin-lightening cleansing composition with a pH below 7.5 comprising:
i. 3% to 50% by weight of detergent active wherein nonionic detergent active, when present, has a HLB in the range 5-18; and
ii. 0.01% to 20% by weight alkyl or cyclo alkyl resorcinol.

2. A skin-lightening cleansing composition as claimed in claim 1 wherein at least 25% by weight of the detergent active is selected from anionic, cationic, amphoteric or zwitterionic surfactants.

3. A skin-lightening cleansing composition as claimed in claim 1 wherein the formulation does not contain any free soap.

4. A skin-lightening cleansing composition as claimed in claim 1 wherein the detergent active is a combination of anionic, nonionic and amphoteric surfactants.

5. A skin-lightening cleansing composition as claimed in claim 1 wherein alkyl resorcinol is selected from ethyl, butyl or hexyl resorcinols.

6. A skin-lightening cleansing composition as claimed in claim 1 comprising 0.1% to 10%, more preferably about 0.1% to about 5% by weight alkyl resorcinol.

7. A skin-lightening cleansing composition as claimed in claim 1 additionally comprising organic or inorganic thickeners.

8. A skin-lightening cleansing composition as claimed in claim 1 comprising 0.01% to 20% by weight organic thickeners selected from crosslinked acrylates (e.g. Carbopol 982), hydrophobically-modified acrylates (e.g. Carbopol 13 82), cellulosic derivatives and natural gums.

9. A skin-lightening cleansing composition as claimed in claim 1 comprising 0.0001% to 80%, preferably from 0.1% to 35%, most preferably from 0.5% to 10% by weight inorganic thickeners selected from thickening silicas, chalk, talc, kaolin, starch, smectite clays, chemically modified magnesium aluminum silicate, organically modified montmorillonite clay, laponite, hydrated aluminum silicate, fumed silica, aluminum starch octenyl succinate.

10. A skin-lightening cleansing composition as claimed in claim 1 comprising 0.01% to 50%, preferably 1% and 15% by weight polyhydric alcohol-type humectants.
